(19) United States
(12) Plant Patent Application Publication
(10) Pub. No.: US 2007/0016986 P1
(43) Pub. Date: Jan. 18, 2007

(54) Raspberry plant named 'MARCELA'

(76) Inventor: Derek L. Jennings, Maidstone (GB)

Correspondence Address: BUCHANAN, INGERSOLL & ROONEY PC, POST OFFICE BOX 1404, ALEXANDRIA, VA, 22313-1404, US

(21) Appl. No.: 11/181,725

(22) Filed: Jul. 15, 2005

(51) Int. Cl. A01H 5/00

(52) U.S. Cl. PLT/204

(57) ABSTRACT

A new and distinct Rubus idaeus L. plant is provided that is the product of a controlled breeding program. The new Raspberry plant abundantly forms attractive conical-shaped Claret Rose fruit which is lighter in coloration than that of the ‘Autumn Bliss’ cultivar (U.S. Plant Pat. No. 6,597) when mature. The new cultivar displays spiney stems and canes unlike the ‘Joan Squire’ cultivar (non-patented in the United States). The fruit is very firm and is firmer than that of the ‘Autumn Bliss’ cultivar, and is well suited for consumption as high grade fresh fruit. The very firm nature of the mature fruit is advantageous during harvest, shipment, and marketing.

### BOTANICAL/COMMERCIAL CLASSIFICATION

Rubus idaeus/Raspberry Plant

### VARIETAL DENOMINATION

cv. ‘Marcela’

### SUMMARY OF THE INVENTION

The instant plant (i.e., Rubus idaeus L.) was created in the course of a planned breeding program carried out at Maidstone, Kent, United Kingdom. Two parents were crossed in the hope that they would contribute the desired characteristics. The female parent (i.e., the seed parent) was the ‘Autumn Bliss’ cultivar (U.S. Plant Pat. No. 6,597). The male parent (i.e., the pollen parent) was the ‘Joan Squire’ cultivar (non-patented in the United States and the subject of European Plant Breeders Rights Grant No. 5680). The ‘Joan Squire’ cultivar is a cross between two hybrids of complex origin. The parentage of the new cultivar can be summarized as follows:
‘Autumn Bliss’×‘Joan Squire’.

The seeds resulting from the above pollination were sown and small plants were obtained which were physically and biologically different from each other. Selective study resulted in the identification of a single plant of the new cultivar.

It was found that the new Raspberry plant of the present invention possesses the following combination of characteristics:
(a) abundantly forms attractive glossy conical-shaped Claret Rose fruit which is lighter in coloration than that of the ‘Autumn Bliss’ cultivar when mature,
(b) displays spiny stems and canes unlike the ‘Joan Squire’ cultivar, and
(c) produces very firm fruit that is firmer than that of the ‘Autumn Bliss’ cultivar and is well suited for consumption as high grade fresh fruit.

The new cultivar well meets the needs of the berry industry. The very firm nature of the mature fruit is advantageous during picking, shipment and marketing.

The new cultivar can be readily distinguished from its ‘Autumn Bliss’ parent in view of its lighter colored mature fruit and its greater fruit firmness. Also, the new cultivar can be distinguished from its ‘Joan Squire’ parent by the presence of spines on the stems and canes which are absent on this parent cultivar.

The new cultivar has been found to undergo asexual propagation at Maidstone, Kent, United Kingdom by in vitro tissue culture and by the rooting of cuttings. Asexual propagation by the above-mentioned methods as performed at such location has shown that the characteristics of the new cultivar are strictly transmissible from one generation to another. Accordingly, the new variety undergoes asexual propagation in a true to type manner.

The new cultivar initially was designated MF941/3, and subsequently has been named ‘Marcela’.

### BRIEF DESCRIPTION OF THE PHOTOGRAPH

The accompanying photograph shows as nearly true as is reasonably possible to make the same in a color illustration of this character a typical specimen of the new cultivar. The photograph was obtained on Jul. 31, 2003 at the Arnold Farm, Maidstone, Kent, United Kingdom. The illustrated plant had been planted during the Spring of 2002.

FIG. 1 illustrates a fruiting plant where the foliage and fruit in various stages of development are illustrated. The attractive Claret Red conical-shaped mature fruit of the new cultivar is shown.

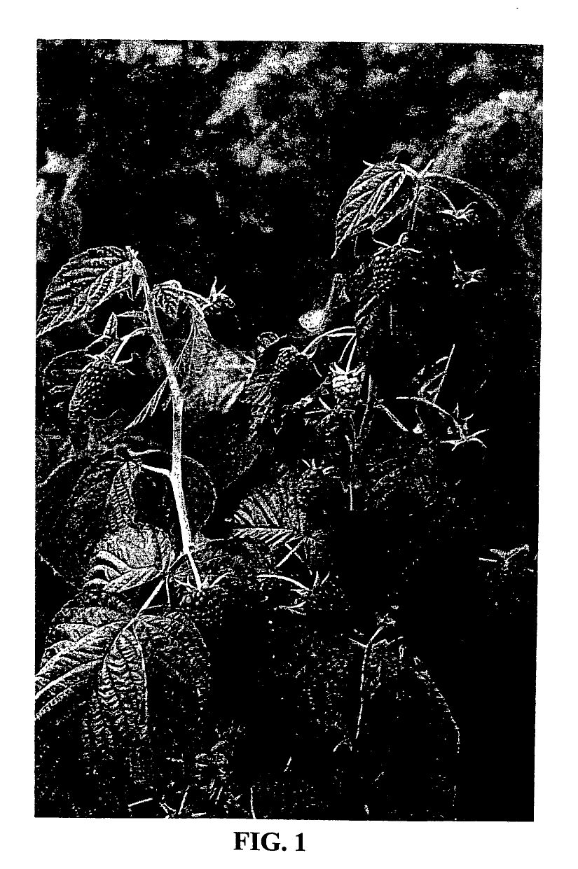

### DETAILED DESCRIPTION

The chart used in the identification of color is that of The Royal Horticultural Society (R.H.S. Colour Chart). The description is based on the observation of specimens of the new cultivar growing at Maidstone, Kent, United Kingdom.
Botanical classification: Rubus idaeus L.
Plant: Displays a generally upright growth habit with a medium number of current season's canes. On a very young rapidly growing shoot anthocyanin coloration is absent at the apex. On a current season's cane anthocyanin coloration is either absent or very weak, the internode length is short, a medium quantity of bloom is present, and the length of the vegetative buds is medium. Also, the length of a current season's cane is medium. The time of cane emergence is generally comparable to that of the ‘Autumn Bliss’ cultivar. Short greenish-brown spines having a small base are sparsely present on the stems and canes.
Foliage: The predominant number of leaflets commonly is approximately equally divided among three and five. The length and width of the terminal leaflets are medium. The leaflets are medium green on the upper surface, generally concave in cross section, and possess weak rugosity. The lateral leaflets are freely positioned. Spines are either absent or very few in number on the leaf pedicels.
Flowering: Begins very early on current season's canes and generally is comparable to the ‘Ariadne’ cultivar (non-patented in the United States) in Southern England. The flower size is medium, and anthocyanin in coloration commonly is absent on the peduncle.
Bearing type: Fruit is mainly borne on the current season's canes. The time of fruit ripening on a current season's cane is very early and generally is comparable to that of the ‘Ariadne’ cultivar, and the length of the fruiting period is medium.
Fruit: Generally conical-shaped in a lateral view and similar in shape to that of the ‘Annamaria’ and ‘Rafzmach’ cultivars (non-patented in the United States). The coloration of the mature fruit is Claret Rose, Red Group 50B, and commonly is lighter than that of its ‘Autumn Bliss’ ancestor. The coloration is similar to that of the ‘Glen Clova’ and ‘Malling Orion’ cultivars (non-patented in the United States). The mature fruit is very firm and is firmer than that of its ‘Autumn Bliss’ ancestor. Such firmness is beneficial during picking, handling, and when transporting to the marketplace. The fruit commonly is borne in small drupes and the individual fruits commonly possess a medium length and width. A strong glossiness is present on the surface of the fruit, and the adherence of the fruit to the plug is medium.
Resistance to pests and diseases: During observations to date the new cultivar of the present invention has displayed adequate resistance to all pests and diseases encountered at Maidstone, Kent, United Kingdom.
Virus status: The virus status of the new cultivar has not been evaluated to date.
Market: The berries are suitable for consumption as a high grade fresh fruit and also are amenable to processing.

I claim:

1. A new and distinct cultivar of Raspberry plant having the following combination of characteristics:
(a) abundantly forms attractive glossy conical-shaped Claret Rose fruit which is lighter in coloration than that of the ‘Autumn Bliss’ cultivar when mature,
(b) displays spiny stems and canes unlike the ‘Joan Squire’ cultivar, and
(c) produces very firm fruit that is firmer than that of the ‘Autumn Bliss’ cultivar and is well suited for consumption as high grade fresh fruit;
substantially as illustrated and described.
